(19) United States
(12) Patent Application Publication
(10) Pub. No.: US 2012/0035560 A1
(43) Pub. Date: Feb. 9, 2012

(54) WOUND TREATMENT SYSTEM

(76) Inventors: Patrick E. Eddy, Allendale, MI (US); Dan J. Parmeter, Ada, MI (US)

(21) Appl. No.: 13/210,805

(22) Filed: Aug. 16, 2011

Related U.S. Application Data
(63) Continuation of application No. 12/936,048.
(60) Provisional application No. 61/041,301, filed on Apr. 1, 2008.

(51) Int. Cl. A61M 1/00

(52) U.S. Cl. 604/313; 604/318; 604/319

(57) ABSTRACT

A system is provided for the treatment of wounds. The system comprises a wound dressing and a suction regulator that includes a vacuum regulator for supplying a negative pressure to the wound site and a control circuit for controlling the vacuum regulator to selectively supply the negative pressure. The system may further comprise (1) a pressure verification mechanism for providing visual verification that the negative pressure applied to the wound site has reached a predetermined pressure level; (2) a venting mechanism for allowing oxygen to vent to the wound site when the negative pressure is not supplied to the wound site; (3) a flow sensor for sensing a flow rate from the wound site where the control circuit generates an alarm signal if the flow rate sensed by the flow sensor exceeds a threshold; and/or (4) a wound dressing pad comprising a support material at least partially covered with silicone.

### CROSS-REFERENCE TO RELATED APPLICATIONS

This application is a continuation of U.S. patent application Ser. No. 12/936,048, which was the National Stage of International Application No. PCT/US2009/039156, filed on Apr. 1, 2009, which claims the benefit of U.S. Provisional Patent Application No. 61/041,301, filed on Apr. 1, 2008, the entire disclosures of which are incorporated herein by reference.

### BACKGROUND OF THE INVENTION

The present invention is generally directed to a system for treating wounds and, more specifically, to a system for treating wounds by applying a vacuum to the wound site.

Wound treatment systems that treat a wound using a vacuum or negative pressure are known. Examples of such systems are disclosed in U.S. Pat. Nos. 4,382,441, 4,392,858, 4,655,754, 4,826,494, 4,969,880, 5,100,396, 5,261,893, 5,527,293, 5,636,643, 5,645,081, 6,071,267, 6,117,111, 6,135,116, 6,142,982, 6,174,306, 6,345,623, 6,398,767, 6,520,982, 6,553,998, 6,814,079, 7,198,046, and 7,216,651. These systems utilize either a manual pump, or a portable vacuum pump to draw air and fluid from the wound site. Such portable pumps can be expensive and take up valuable space in the hospital recovery rooms.

A related commonly-assigned published U.S. Patent Application Publication No. US 2009/0043268 A1 discloses a wound treatment system that may use an existing vacuum system of a hospital or healthcare facility, either remote or central.

### SUMMARY OF THE INVENTION

According to one aspect of the present invention, a system is provided for the treatment of wounds by applying a negative pressure to a wound site. The system comprises a suction regulator and a wound dressing. The suction regulator comprises a vacuum regulator for supplying a negative pressure to the wound site, and a control circuit coupled to the vacuum regulator for generating control signals for controlling the vacuum regulator to selectively supply the negative pressure to the wound site. The wound dressing is provided at the wound site and coupled to the vacuum regulator. The wound dressing comprises a wound dressing pad for placing over the wound, and a wound drape provided over the wound dressing pad and the wound site for securing the wound dressing pad and sealing the wound site for application of the negative pressure. The system further comprises a pressure verification mechanism in communication with the wound site for providing visual verification that the negative pressure applied to the wound site has reached a predetermined pressure level.

According to another aspect of the present invention, a system is provided for the treatment of wounds by applying a negative pressure to a wound site. The system comprises a wound dressing and a suction regulator. The suction regulator comprises a vacuum regulator for supplying a negative pressure to the wound site, and a control circuit coupled to the vacuum regulator for generating control signals for controlling the vacuum regulator to selectively supply the negative pressure to the wound site. The wound dressing is provided at the wound site and coupled to the vacuum regulator. The wound dressing comprises a wound dressing pad for placing over the wound, and a wound drape provided over the wound dressing pad and the wound site for securing the wound dressing pad and sealing the wound site for application of the negative pressure. The system further comprises a venting mechanism for allowing oxygen to vent to the wound site when the negative pressure is not supplied to the wound site.

According to another aspect of the present invention, a system is provided for the treatment of wounds by applying a negative pressure to a wound site. The system comprises a wound dressing and a suction regulator. The suction regulator comprises: a vacuum regulator for supplying a negative pressure to the wound site, a flow sensor for sensing a flow rate from the wound site, and a control circuit coupled to the flow sensor and to the vacuum regulator for generating control signals for controlling the vacuum regulator, the control circuit generating an alarm signal if the flow rate sensed by the flow sensor exceeds a threshold. The wound dressing is provided at the wound site and coupled to the vacuum regulator. The wound dressing comprises a wound dressing pad for placing over the wound, and a wound drape provided over the wound dressing pad and the wound site for securing the wound dressing pad and sealing the wound site for application of the negative pressure.

According to another aspect of the present invention, a system is provided for the treatment of wounds by applying a negative pressure to a wound site. The system comprises a wound dressing and a suction regulator. The suction regulator comprises a vacuum regulator for supplying a negative pressure to the wound site, and a control circuit coupled to the vacuum regulator for generating control signals for controlling the vacuum regulator to selectively supply the negative pressure to the wound site. The wound dressing provided at the wound site and coupled to the vacuum regulator. The wound dressing comprises a wound dressing pad for placing over the wound, and a wound drape provided over the wound dressing pad and the wound site for securing the wound dressing pad and sealing the wound site for application of the negative pressure. The wound dressing pad comprises a support material at least partially covered with silicone.

According to another aspect of the present invention, a wound dressing pad is provided for use in a system for the treatment of wounds by applying a negative pressure to a wound site. The wound dressing pad comprises a support material at least partially covered with silicone for application to a wound at the wound site.

These and other features, advantages, and objects of the present invention will be further understood and appreciated by those skilled in the art by reference to the following specification, claims, and appended drawings.

### BRIEF DESCRIPTION OF THE DRAWINGS

In the drawings:

FIG. 1A is a perspective view of a portion of a wound treatment system according to one embodiment of the present invention;

FIG. 1B is a perspective view of a portion of a wound treatment system according to another embodiment of the present invention;

FIG. 2 is a fluid flow and electrical circuit diagram in block form of a wound treatment system according to the present invention;

FIG. 3 is an electrical circuit diagram in block form of a suction regulator according to the present invention;

FIG. 4 is a perspective view of a wound dressing portion that may be used in the inventive wound treatment system;

FIG. 5 is a cross-sectional view of the wound dressing portion shown in FIG. 4 taken along line 2-2;

FIG. 6 is a top view of the wound dressing portion shown in FIG. 4;

FIG. 7 is a cut-away perspective view of a portion of the bottom surface of a drape of the wound dressing portion shown in FIGS. 4 and 6;

FIG. 8 is a plan view of the bottom surface of an attachment pad that may be used in the inventive wound treatment system;

FIG. 9 is a side view of the attachment pad shown in FIG. 8;

FIG. 10 is a side view of the attachment pad shown in FIG. 8 shown when in use in the inventive wound treatment system;

FIG. 11 is a perspective view of a wound dressing portion configured for attachment to a patient's leg;

FIG. 12A is a cross-sectional view of an example of a wound dressing pad that may be used in the inventive wound treatment system; and

FIG. 12B is a cross-sectional view of another example of a wound dressing pad that may be used in the inventive wound treatment system.

### DETAILED DESCRIPTION OF THE PREFERRED EMBODIMENTS

Reference will now be made in detail to the presently preferred embodiments of the invention, examples of which are illustrated in the accompanying drawings. Wherever possible, the same reference numerals will be used throughout the drawings to refer to the same or like parts.

For purposes of description herein, the terms “upper,” “lower,” “right,” “left,” “rear,” “front,” “vertical,” “horizontal,” “top,” “bottom,” and derivatives thereof shall relate to the invention as shown in the drawings. However, it is to be understood that the invention may assume various alternative orientations, except where expressly specified to the contrary. It is also to be understood that the specific devices illustrated in the attached drawings and described in the following specification are simply exemplary embodiments of the inventive concepts defined in the appended claims. Hence, specific dimensions, proportions, and other physical characteristics relating to the embodiment disclosed herein are not to be considered as limiting, unless the claims expressly state otherwise.

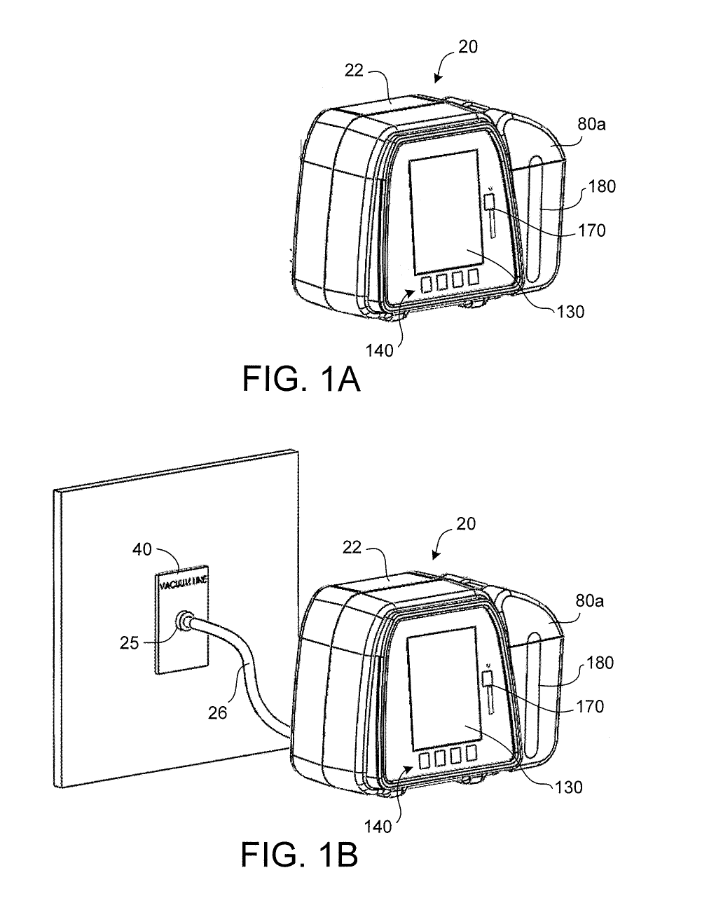

As shown in FIGS. 1A and 1B, a system is provided for treatment of wounds that includes an electronically controlled suction regulator 20. According to a first variation shown in FIG. 1A, the regulator 20 includes an internal vacuum pump. According to a second variation shown in FIG. 1B, the regulator 20 that connects to an external vacuum source 40. Such an external vacuum source may include the built-in central vacuum system of a healthcare facility, central vacuum pump remotely located from the suction regulator, or a separate portable vacuum pump. Such a connection may be via an appropriately configured coupler 25 and a supply hose 26. This system may be used for numerous health provider procedures and devices. As described further below, this system may have special safety features built in to protect the patient. The term “built-in” vacuum system is intended to refer to vacuum systems that are plumbed into the building structure of a healthcare facility and is not intended to cover a vacuum pump mounted to a wall or other structure of the patient's room. “Healthcare facility” is intended to include hospitals, outpatient treatment facilities, doctors' offices, nursing homes, and any other facility in which healthcare services are provided.

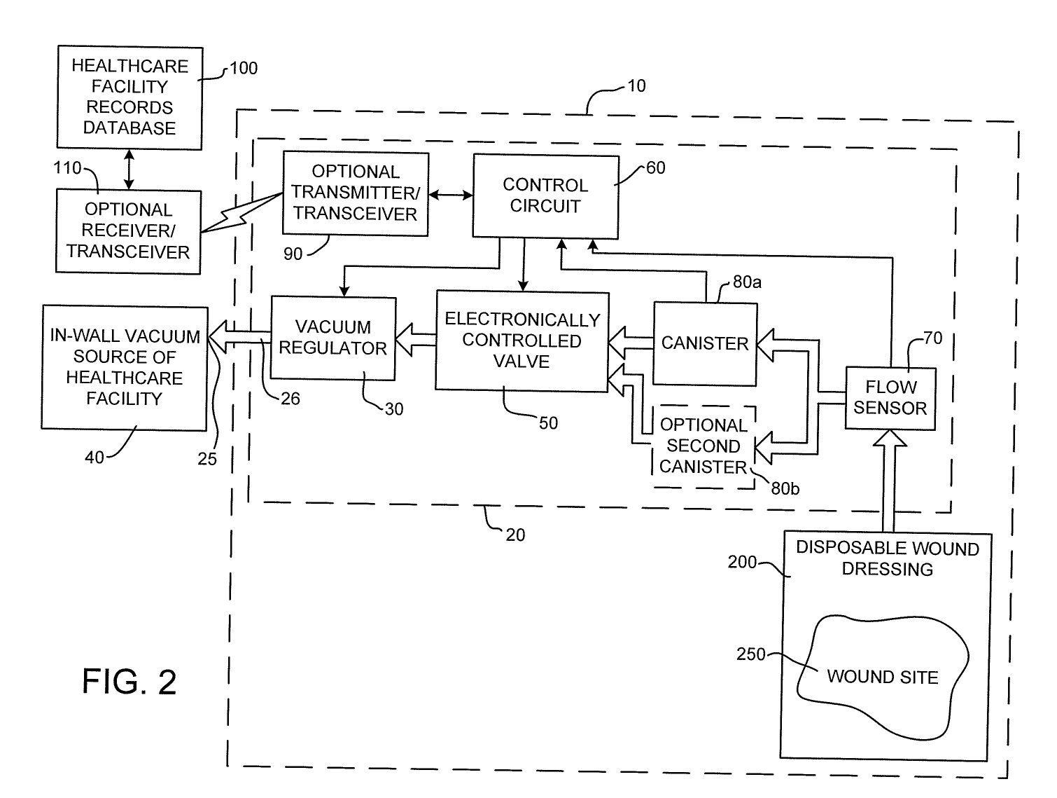

As shown in FIGS. 1A, 1B, and 2, the system 10 may include electronically controlled suction regulator 20 as a single unit having a housing 22 with integrated regulation of the vacuum and a containment apparatus 80a that will contain solids and liquids, but let gaseous materials pass to the atmosphere. The purpose is to provide a safe method of providing either constant or intermittent/modulated vacuum to a physician or health provider for use on a patient or connection to a device that may or may not be used on a patient. This device is electronically controlled and will perform various functions including the ability to lock-out users from changing settings to alarming functions for safety and efficacy.

As shown in FIG. 2, system 10 may further include a disposable wound dressing 200 for application to a wound site 250 of a patient. As discussed further below, dressing 200 effectively seals the wound site so that a negative pressure may be maintained at the wound site.

As shown in FIG. 2, electronically controlled suction regulator 20 may comprise any one or more of the following: a vacuum regulator 30 that is either a vacuum pump or is otherwise connected to either the vacuum system 40 of the healthcare facility or an external portable vacuum pump; a valve or valve type system 50 (such as an electronically controlled three-way solenoid valve or a pneumatic valve) connected to vacuum regulator 30; a control circuit 60 powered by electrical current for controlling various components of the regulator and for generating control signals for controlling valve 50 so that the source of vacuum supplied to a patient or device for predetermined periods of time is able to deliver constant or intermittent vacuum; a flow sensor 70, such as a pressure transducer, connected to control circuit 60 for monitoring the negative pressure applied to a patient or device; a canister/basin 80a for collecting fluids drained from the patient's wound; an optional second canister/basin 80b for collecting additional fluids drained from the patient's wound; and an optional transmitter or transceiver 90 for transmitting information to a healthcare facility database 100 via an optional receiver or transmitter 110.

The source of vacuum may have a vacuum between 0 and 600 mmHg, and may be either vacuum pump internal to housing 22 or an external vacuum system 40 built into a healthcare facility, such as a distributed hospital vacuum system. The vacuum from system 40 may be regulated by vacuum regulator 30 operating under control of control circuit 60 and may be selectively applied continuously or intermittently or may be interrupted by valve 50. The application of negative pressure to the wound site 250 can be actuated at predetermined time intervals or in response to wound site conditions such as an accumulation of fluid under the wound dressing 200. During an intermittent vacuum mode the apparatus may vent to the ambient atmosphere or supply low pressure oxygen to the wound during vacuum off time.

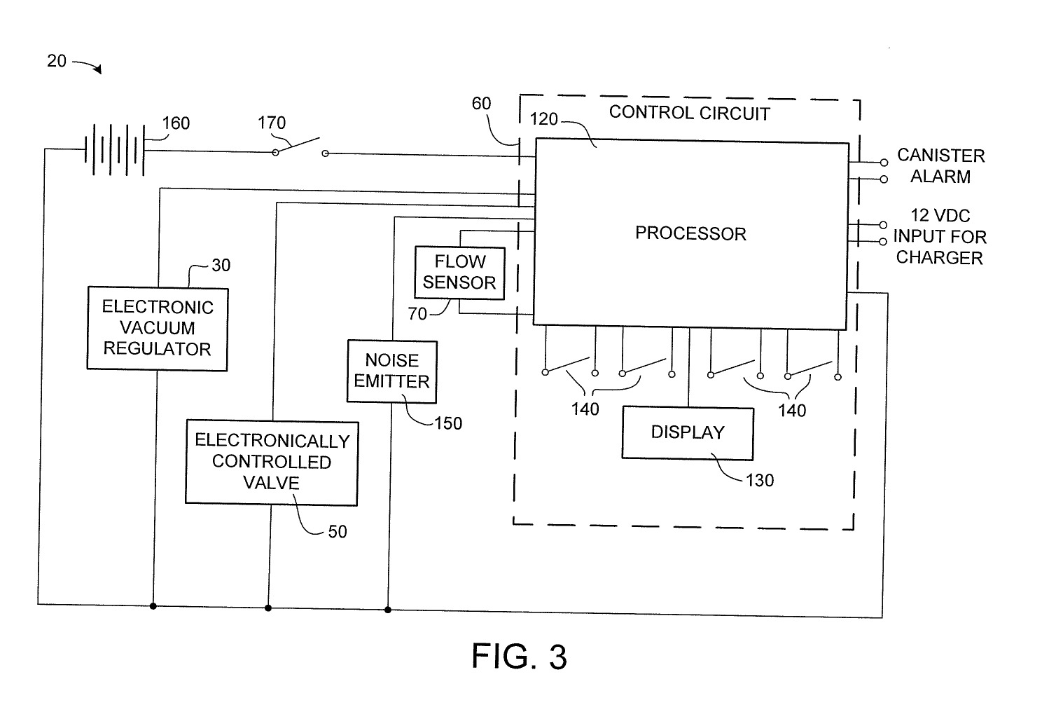

As shown in FIG. 3, control circuit 60 may comprise a programmable digital processor 120 and a liquid crystal display or similar technology display panel 130 connected to the other electronic circuitry. Control circuit 60 may further include an end user interface 140 such as a touch pad with one or more switches, connected to processor 120. Processor 120 may be programmable to turn the electronics on and off at prescribed times. In addition, end user interface may be configured to allow an end user to select various settings that may be employed to adjust the characteristics (i.e., timing cycle, intermittent mode, continuous mode, pressure, etc.) of the suction produced at the output of suction regulator 20. In addition, The electronically controlled suction regulator may provide the ability to lock out negative pressure settings so that the patients cannot change settings by the healthcare providers.

Suction regulator 20 may further comprise a rechargeable battery 160 and a main power switch coupled in series with the control circuit 60 so as to selectively power the portable device. Regulator 20 may also include a pair of terminals for connection to a 12 VDC input for charging the battery. Control circuit 60 may include an AC to DC converter and regulating circuitry that may be connected to these terminals such that regulated DC power is supplied to the electronic circuitry and the battery 160.

Canister/basin 80a may have an adjustable proximity switch connected to processor 120 for generating an audible using a noise emitter 150 and/or a visual alarm using an LED or LCD 130 to indicate that the contents have reached a particular level. Canister/basin 80a may be used with a ‘gel pack’ and or a porous filter. Preferably, canister/basin 80a includes he fluid level sensor disclosed in U.S. patent application Ser. No. 12/262,474, entitled “FLUID LEVEL SENSOR FOR A CONTAINER OF A NEGATIVE PRESSURE WOUND TREATMENT SYSTEM,” filed on Oct. 31, 2008 by Albert A. Schenk III et al., the entire disclosure of which is incorporated herein by reference.

Housing 22 and canister 80a may be made from polymers for light weight and impact resistance. Further, canister/basin 80a may be replaceable and thus disposable and may contain about 250-1500 ml. Canister/basin 80a may be removable and may be sealed with a gasket, o-ring, or similar sealing apparatus. Canister/basin 80a may be frosted to obstruct portions of view but is clear in specific areas 180 to view contents and compare to a scale such as but not limited to ml. Canister/basin 80a may be a portion less than a ¾ circle but more than a ¼ circle and may be keyed to fit the unit 20 with an integral incorporated into basis conduit/hose with a press fit cradle.

Electronically controlled suction regulator 20 may thus comprise a safe regulation system with integrated (basin/canister/reservoir) and device for preventing liquids from leaving the (basin/canister/reservoir) thus containing possible contaminates. Further, the electronically controlled suction regulator may comprise integrated electronics that will regulate between 0 and 600 mmHg and provide ability to modulate/intermittent between negative pressure and atmospheric pressure. The electronically controlled suction regulator may have a mechanical method for determining fluid level in canister 80a and the ability to stop the vacuum.

Although the application described herein of suction regulator 20 is that of negative pressure wound treatment (NPWT), suction regulator 20 may be used in a variety of applications. The electronically controlled suction regulator is well-suited for use in healthcare facilities as a general safe method of filtering and regulating reduced pressure for procedures such as but not limited to: nasopharyngeal, tracheal, surgical, gastrointestinal, pleural, wound drainage, etc. The features that make suction regulator 20 uniquely suited for NPWT is its ability to: (1) allow end user adjustment of the output suction characteristics (i.e., timing cycle, intermittent mode, continuous mode, pressure, etc.), (2) generate an alarm if fluid in a canister reaches a particular level, and (3) generate an alarm if the flow rate from the wound is too high (above a threshold level), which indicates a leak.

The electronically controlled suction regulator 20 may be hung on a wall using preexisting brackets or may be placed on a bed using a clamp or pole, or be free standing with and without an optional base and an IV pole.

A filter/fluid trap that is permeable by gas only and not permeable by solids or liquids may be interposed between the vacuum source and the canister/basin to prevent solids or liquids from being introduced into the regulator system, the conduits, or the vacuum source. The filter may be a porous polymer that impedes solids and liquids from passing but allows gaseous materials to pass. The filter may be a polymer or other natural substance. The filter may be single or plural but may cover all conduits exiting reservoir/canister. An outlet conduit for fluid may be connected between an outlet port of the canister and the vacuum source, and the filter may be disposed in the canister substantially at the interface between the outlet port and the outlet conduit.

A first pressure detector may be provided that is adapted to detect a pressure drop indicative of the filter being substantially covered/blocked by water or solids.

The suction regulator may further comprise an optional negative pressure detector disposed in the inlet conduit that may compare the measured pressure with a preset level to determine if the negative pressure/vacuum is at or above the preset pressure level. This system will work with a single conduit/tube and can aid in prevention of blockage without need for separate detection systems.

The suction regulator electronics may be configured to time stamp the proximity switches position by operator. As explained further below, such time stamps and switch positions may be supplied to the healthcare facility's records database.

The electronics logic may be configured to protect patients by alarming if too much fluid is contained in the canister in a pre-entered time frame.

According to one embodiment of the present invention, the regulator system 10 is used for applying a negative pressure to a wound. This may be accomplished by connecting the outlet conduit of the suction regulator 20 to the patient interface portion 200 of the systems disclosed in U.S. Pat. Nos. 4,382,441, 4,392,858, 4,655,754, 4,826,494, 4,969,880, 5,100,396, 5,261,893, 5,527,293, 5,636,643, 5,645,081, 6,071,267, 6,117,111, 6,135,116, 6,142,982, 6,174,306, 6,345,623, 6,398,767, 6,520,982, 6,553,998, 6,814,079, 7,198,046, and 7,216,651, the entire disclosures of which are incorporated herein by reference.

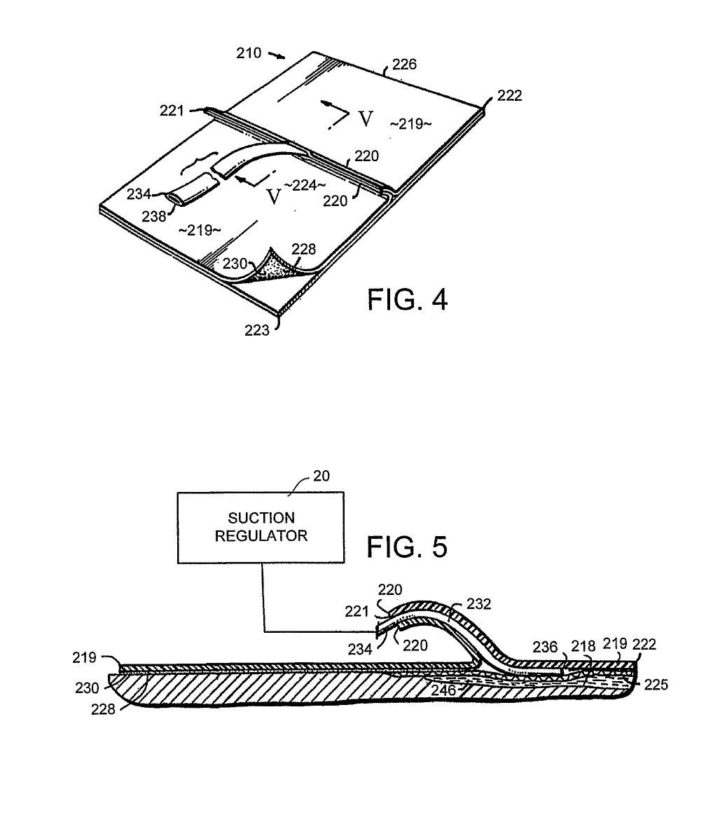

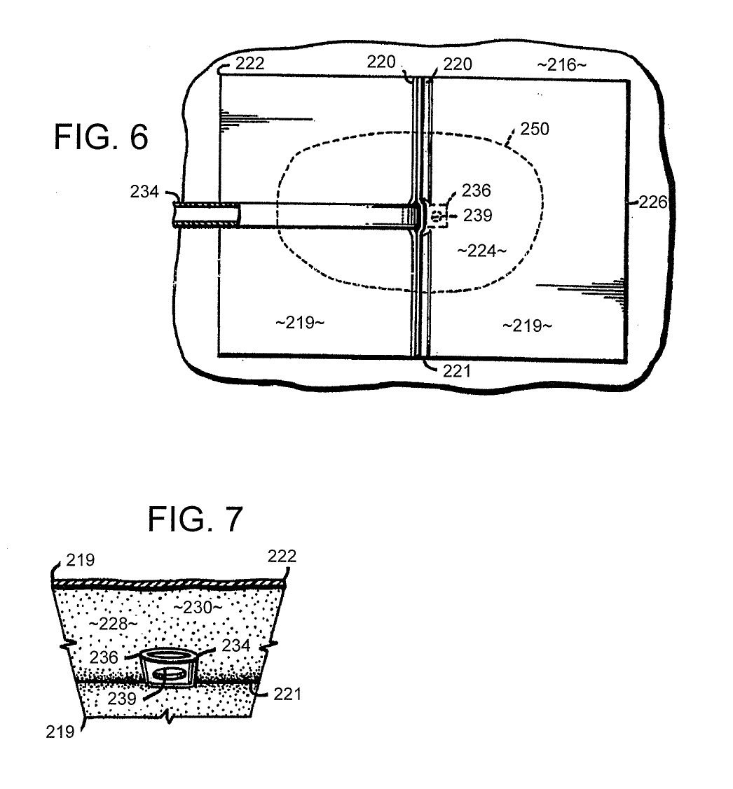

FIGS. 4-12B relate to disposable wound dressings 200 that may be used in the inventive system. The disposable wound dressings shown in FIGS. 4-12B are described below. FIGS. 4-7 show an example of one wound treatment system to which the various improvements may be implemented separately or in various combinations. FIG. 4 is a perspective view of a disposable wound dressing 200. Disposable wound dressing 200 includes wound drape 222 that includes an interior portion 224 surrounded by a perimeter 226. Drape 222 further includes a skin contact surface 228 with an adhesive coating 230. The drape may be made of membrane permeable, semi-permeable or non-permeable materials that are commercially available, an example being material referred to as TAGODERM®, which is available from the 3M (Minnesota Mining and Manufacturing) Company of St. Paul, Minn. A protective backing 223 is placed over the adhesive coating 230 on the skin contact surface 228 until drape 222 is ready for application.

Wound drape 222 may comprise a pair of panels 219 with inner, upturned edges 220 which can be adhesively joined together to form a seam 221 which extends transversely across drape 222 and projects generally upwardly therefrom. The panels 219 can be secured together at the seam 221 by the adhesive coating 230 to form the seam 221. Alternatively, drape 222 may be made of a single panel as described further below.

The vacuum conduit may include a tube or sheath 234 that includes a proximate end 36 located under drape 222 and a distal or free end 238. The tube 234 can be inserted through the seam 21 which forms an opening 232 between the panel edge strips 220 at approximately the center of the drape 222. If a single panel 219 is used such that no seam is present, a hole may be formed in the drape 222 for passage of the tube or for placement of an attachment pad or coupler (discussed below). A relatively short length of the tube 234 adjacent to its proximate end 236 is shown under the drape 222 in FIG. 5, but greater lengths of the tube 234 could be placed under the drape 222. As shown in FIG. 7, the tube proximate end 36 is open, and adjacent to the proximate end 236 one or more openings are formed. The tube opening(s) 239 may project downwardly, i.e. away from the skin contact surface 228. The short length of the tube 234, which is located under drape 222, can be releasably secured to the skin contact surface 228 by the adhesive coating 230, preferably with the tube opening 239 facing downwardly. The tube 234 may have a length that is sufficient to extend to the vacuum source 242 or to the containment apparatus 241. Alternatively, a second tube may be attached to the free end 238 of the tube 234.

The tube 234 can comprise, for example, a flexible, plastic tube of the type that is commonly used as a percutaneous sheath for intravenous treatments. At its distal end 238, the tube 234 may be adapted for: (1) closure with a variety of suitable closure devices; (2) connection to various active and passive fluid collection devices for draining and evacuating fluid from the wound site; and (3) connection to various fluid source devices for actively and passively introducing fluid to the wound site.

FIG. 5 shows the tube distal end 238 fluidically communicating with a suction regulator 20 for actively draining fluid from the wound site.

The disposable wound dressing 200 may further include a wound dressing pad 225 between the wound site 250 and drape 222. The wound dressing pad 225 can comprise a variety of materials with varying properties such as: (1) absorbency; (2) wicking or capillary action; and (3) surface contact action. The wound dressing 225 is primarily located in a chamber 246 formed between the wound 250 and the drape 222.

In wound treatment systems such as the one described above, the wound dressing pad 225 is sized and shaped to fit in and over the wound to be treated, and thus the wound dressing is in direct contact with the wound. In prior systems, a gauze or foam is used as the wound dressing pad so as to allow air to flow around the wound. The air flow is caused by the application of a vacuum. Because the vacuum also tends to draw fluids from the wound and through the wound dressing pad, the wound tissue can grow into the wound dressing pad or otherwise stick to the wound dressing pad. This causes problems in that the wound does not heal properly and can also reopen when the wound dressing is removed or changed. In addition, the removal of a wound dressing pad that is stuck to the wound, can be particularly uncomfortable for the patient.

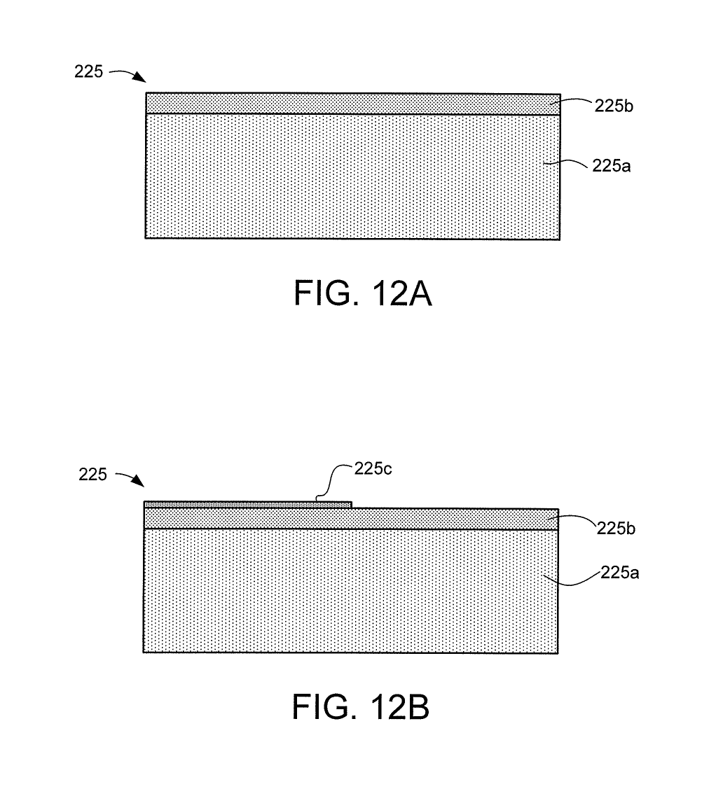

Wound dressing pad 25 may be siliconized to allow tissue on and around the wound to form without growing into or onto the wound dressing pad or from otherwise sticking to the wound tissue. The wound dressing pad may include a support material such as a natural fiber, polymer, foam (such as a granufoam-urethane base or whitefoam-PVA base), or other filler/support material. An example of a foam is a granufoam available from Kinetic Concepts, Inc. (KCI) of San Antonio, Tex. The filler/support material could be “siliconized.” This can occur by at least partially covering the support material with silicone which can be accomplished by applying silicone to at least the surface of the filler/support material that directly contacts the wound, by impregnating the filler/support material with silicone, or by using a filler/support material that already integrally includes silicone or its equivalent. By using silicone or an equivalent, the wound can properly heal without the wound tissue growing into or sticking to the wound dressing pad. One commercially available material that may be used as the wound dressing is THERAGAUZE®, which is available from Soluble Solutions, LLC of Newport News, Va. The formulation of THERAGAUZE® is believed to be disclosed in U.S. Pat. No. 6,592,860, the entire disclosure of which is incorporated herein by reference. Alternatively, one may use foam that is seared to close cells on the foam surface adjacent the wound, or use a dual-density foam (two styles of foam together for different end effects) as shown in FIGS. 12A and 12B. Specifically, the dual density foam pad 225 includes a larger cell foam layer 225a and a smaller cell foam layer 225b that contacts the wound. As shown in FIG. 12B, the foam pad 225 may further include an optional coating 225c of a material such as silicone.

The silicone/seared foam may or may not be perforated or slit to allow vacuum, ambient or a positive pressure to pass through, and to allow liquids to pass. Whether to perforate or slit the silicone will depend upon the particular application and the nature of the filler/support material and how the silicone is provided.

The siliconized wound, seared, dual-density dressing pads 225 may be coated with a medicated or non-medicated solution such as polypropylene, glycol and saline, silver, an anti-bacterial solution or the like, that may promote healing and/or reduce adhesion of tissue and fluids.

Alternatively, wound dressing pad 225 is made of a bio-absorbable material such that wound tissue growth into pad 225 because a positive condition rather than a negative condition as the pad may simply be left in place into the patient's body absorbs the pad.

The wound drape 222 may be any conventional drape material known to be used for vacuum-assisted wound treatment. The material may be a semi-permeable or impermeable flexible covering that may or may not have a valve/relief to the outside atmosphere. The wound drape may have one or more apertures for allowing a tube, attachment pad, or other coupler to be inserted for connection of the vacuum conduit and application of the vacuum to the wound. The application of the vacuum may be regulated and varied during a course of treatment. In addition, the vacuum may be intermittently applied.

The system may use a tube that has a plurality of apertures through its sidewalls at the end of the tube that extends into and under the wound drape. The end of the tube may lie between the drape and the wound dressing or it may extend into the wound dressing.

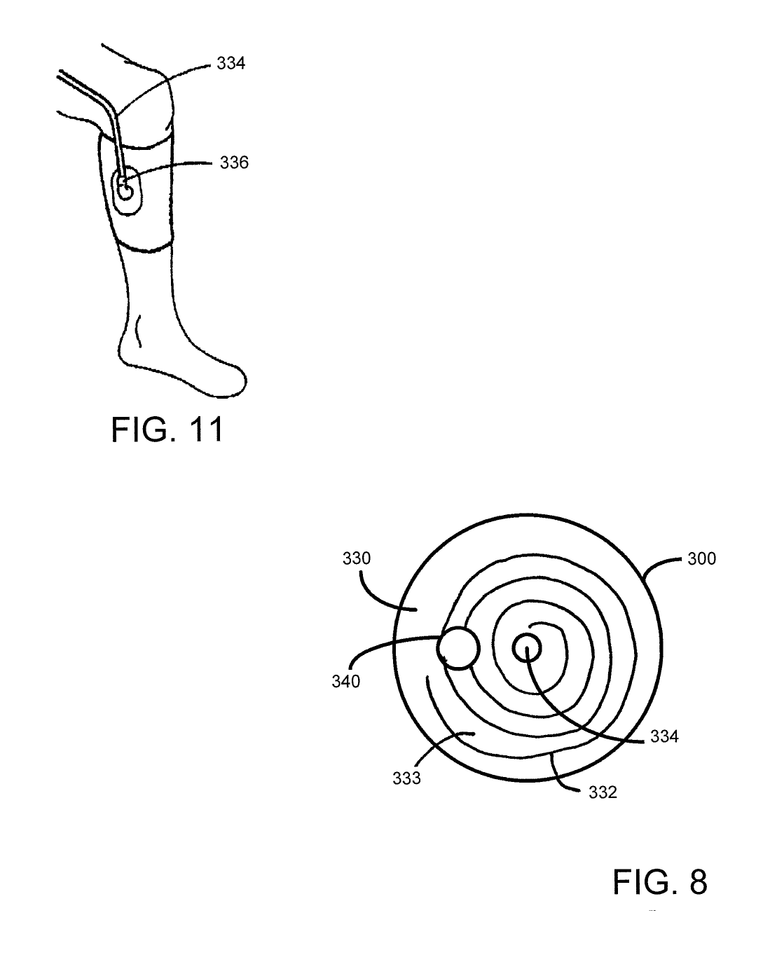

An attachment pad/coupler has been developed that includes pressure verification mechanism that may be a mechanical device to provide a visual acknowledgement of vacuum at a predetermined pressure level at or near the wound site. In general, an attachment pad/coupler 300 such as that shown in FIGS. 8-11, comprises a flange portion 330 having a tapered edge 331, and a profile which may be of any desired shape. On the face of the flange 330 that intended for contact with the wound dressing pad 225 are one or more projections 332. The purpose of these projections is to provide one or more fluid channels 333 facilitating the flow of fluids form any point of the flange to a central aperture 334, from which it is intended to apply suction. The attachment pad 300 includes a connector 335, located above the aperture 334, having a tubular end 336 adapted for receiving and connecting to the vacuum conduit. The tubular and may have an outwardly tapered portion to facilitate feeding a tube into the connector. The upper surface 337 of the attachment pad 300 has a substantially smooth surface with the exception of at least one bubble or dome 340 (described further below). Linear attachment may be used in lieu of the attachment pad/coupler.

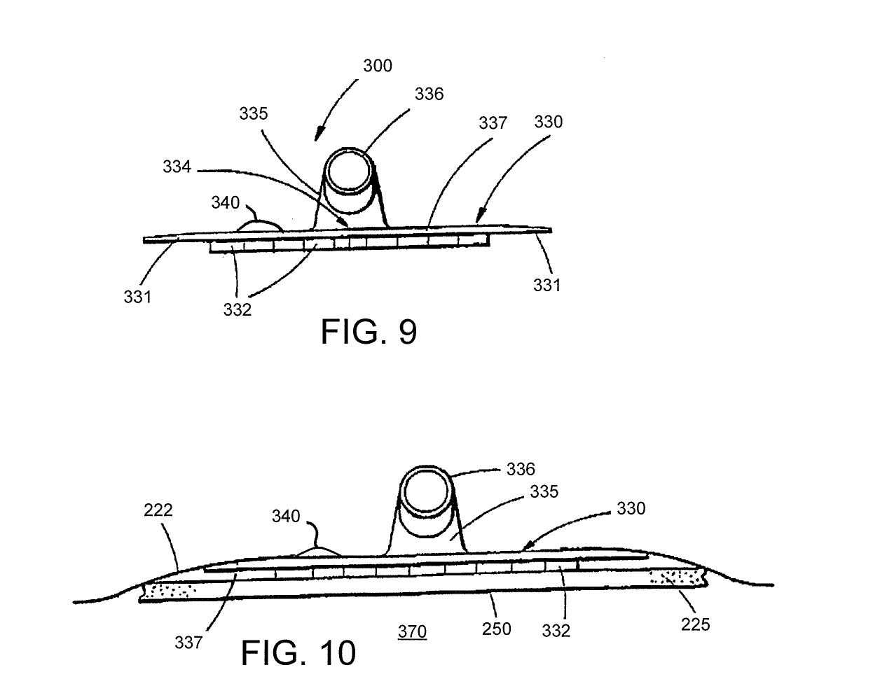

In use, the connector portion 335 is sized so that it extends through the aperture 325 in the wound drape 222 shown in FIGS. 9 and 10, with the adhesive surface around the aperture bonded to the smooth surface 337 of the flange 330. The flange 330 of the attachment pad 300 may be circular as shown in FIG. 11. Alternatively, the flange may be any other shape.

FIGS. 10 and 11 show the attachment pad 300 attached to a wound site 250 of a patient 370. The attachment pad 300 is pressed into firm contact with wound dressing pad 225, which is itself pressed into contact with a wound area 250. The attachment pad 300 and wound dressing pad 225 are pressed into contact with the wound area by a wound drape 222. The adhesive surface 330 of drape 222 is bonded to the patient's skin outside the periphery of the wound dressing pad 225 and attachment pad 300. It is also bonded to upper surface 337 of the attachment pad 300. Aperture 325 is formed in the drape 222 to permit the connector portion 335 to extend upwardly through the drape.

As mentioned above, attachment pad 300 has at least one convex bubble or dome 340 formed in one of its surfaces, that is sucked inward increasing vacuum pressure at our near the wound site 250. The size, thickness, and material used for the bubble or dome could be used to calculate an approximate vacuum recognition that would be changeable in the mold itself. The attachment pad 300 could include multiple bubbles that each indicating different vacuum levels such as 50, 100, and 150 mm Hg.

An attachment pad such as those disclosed in U.S. Pat. Nos. 6,345,623, 6,553,998, and 6,814,079 may also be used with the inventive system. In addition, a TRACKPAD™ available from KCI may also be employed.

As noted above, the wound treatment system 10 may include two canisters 80a and 80b (FIG. 2). Existing systems use a single canister that has an alarm that is triggered when the canister becomes full. When the canister becomes full, the vacuum system is stopped until the healthcare professional overseeing the treatment of the patient, can get to the room, remove and empty the canister, return the canister, and restart the system. All of this takes time and interrupts the procedure. By using two canisters, a first canister can be used in the normal course, and when the alarm is generated, a signal is sent to an electronically controlled valve that diverts the flow of fluid from the first canister 80a to the second canister 80b to thereby allow uninterrupted use. When the alarm is generated indicating the first canister 80a is full, the healthcare professional overseeing the treatment of the patient, can empty the first canister as was done previously, except that the system can keep operating with the fluid flowing to the second canister 80b. Upon returning the empty first canister 80a, the system can either automatically return the flow of fluid to the first canister 80a or continue the flow of fluid to the second canister 80b until such time that it becomes full—at which time the valve may be reactuated to divert the flow to first canister 80a.

The level of fluid in the canisters 80a, 80b may be monitored using a continuity sensor that includes two electrically conductive terminals spaced apart at the upper internal region of the canisters such that current flows from one terminal to the other only when the fluid level reaches the terminals thereby causing an alarm.

Flow sensor 70 may be used to monitor the pressure of the vacuum and determines if a predetermined start up pressure lasts for a certain time. This feature (also known as “wound close technology”) allows one to monitor the progression of the wound to closure. This can be displayed on display screen 130 and would work as an initial start cycle function that can be done at a new wound site, change of dressing, or as a special cycle that will work when the wound site is at ambient/atmospheric pressure.

A venting mechanism may be provided at the attachment pad, in the suction regulator or elsewhere that allows oxygen from ambient air to be vented to the wound at 1 or 2 psi whenever the vacuum is in an off interval of an intermittent cycle or the vacuum is removed. Alternately, an oxygen source may be provided to supply oxygen during such off periods. The venting mechanism may comprise valve 50, which can be selectively controlled to alternatingly connect either vacuum regulator 30 or ambient air (or an oxygen source) to the wound dressing.

The system may also be configured to a high flow (leak detection) alarm that is activated when the flow of air from the wound site is above a threshold.

Referring back to FIG. 2, optional transmitter/transceiver 90 may be provided to transmit information to a receiver/transceiver 110 that receives the information and provides it to an automated records database 100 of the healthcare facility. The information may include any one or more of the following: the times at which negative pressure was applied to the wound, the pressure applied, the intermittence cycles, the times at which the settings were changed along with the new settings, leak detection alarm times, full canister alarms times, and readings from flow sensor 70 which allows one to monitor the progression of the wound to closure. Transmitter/transceiver 90 may be coupled wirelessly or by wired connection such as USB. The database 100 may be a database such as a Cerner records database.

Each of the above-noted features may be implemented separately from the other features, or in combination with one or more of the other features.

The above description is considered that of the preferred embodiments only. Modification of the invention will occur to those skilled in the art and to those who make or use the invention. Therefore, it is understood that the embodiments shown in the drawings and described above are merely for illustrative purposes and not intended to limit the scope of the invention, which is defined by the following claims as interpreted according to the principles of patent law, including the Doctrine of Equivalents.

What is claimed is:

1. A system for the treatment of wounds by applying a negative pressure to a wound site, the system comprising:
a suction regulator comprising:
a vacuum regulator for supplying a negative pressure to the wound site, and
a control circuit coupled to said vacuum regulator for generating control signals for controlling said vacuum regulator to selectively supply the negative pressure to the wound site;
a wound dressing provided at the wound site and coupled to said vacuum regulator, said wound dressing comprising:
a wound dressing pad for placing over the wound, and
a wound drape provided over said wound dressing pad and the wound site for securing said wound dressing pad and sealing the wound site for application of the negative pressure; and
a pressure verification mechanism in communication with the wound site for providing visual verification that the negative pressure applied to the wound site has reached a predetermined pressure level.

2. The system of claim 1, wherein said system further comprises an attachment pad and a vacuum conduit coupled at one end to said suction regulator and at another end to said attachment pad, said attachment pad provided at the wound dressing and configured to apply the negative pressure from said conduit to the wound site.

3. The system of claim 2, wherein said attachment pad comprises a flange.

4. The system of claim 3, wherein said pressure verification mechanism comprises at least one dome formed in said flange, where the dome extends outward from said flange when the negative pressure has not reached the predetermined pressure level and collapses once the negative pressure has reached the predetermined pressure level.

5. The system of claim 1, wherein said control circuit generates control signals for controlling said vacuum regulator so that negative pressure may be continuously or intermittently supplied to the wound site.

6. The system of claim 1, and further comprising:
a canister operatively coupled to said vacuum regulator for receiving and storing fluids drawn from the wound.

7. The system of claim 6 and further comprising:
a fluid level alarm provided in said canister for supplying a fluid level alarm signal to said control circuit when said canister is full of fluid.

8. The system of claim 7 and further comprising:
a second canister operatively coupled to said vacuum regulator for receiving and storing fluids drawn from the wound, wherein said control circuit controls an electronically controlled valve to apply the vacuum drawn by said vacuum regulator to said second canister when said fluid level alarm signal is received.

9. The system of claim 1, and further comprising a flow sensor for sensing a flow rate from the wound site, wherein an alarm is sounded if the flow rate exceeds a threshold.

10. The system of claim 1, wherein said wound dressing pad is bio-absorbable.

11. A system for the treatment of wounds by applying a negative pressure to a wound site, the system comprising:
a suction regulator comprising:
a vacuum regulator for supplying a negative pressure to the wound site, and
a control circuit coupled to said vacuum regulator for generating control signals for controlling said vacuum regulator to selectively supply the negative pressure to the wound site;
a wound dressing provided at the wound site and coupled to said vacuum regulator, said wound dressing comprising:
a wound dressing pad for placing over the wound, and
a wound drape provided over said wound dressing pad and the wound site for securing said wound dressing pad and sealing the wound site for application of the negative pressure; and
a venting mechanism for allowing oxygen to vent to the wound site when the negative pressure is not supplied to the wound site.

12. The system of claim 11, wherein said venting mechanism comprises a valve of said suction regulator where said valve applies either oxygen or the negative pressure to the wound site in response to control signals from said control circuit.

13. The system of claim 12, wherein said valve selectively couples one of the vacuum regulator and an oxygen source to said wound dressing.

14. The system of claim 11, wherein said venting mechanism vents to an ambient atmosphere to allow oxygen in the ambient atmosphere to reach the wound site.

15. A system for the treatment of wounds by applying a negative pressure to a wound site, the system comprising:
a suction regulator comprising:
a vacuum regulator for supplying a negative pressure to the wound site,
a flow sensor for sensing a flow rate from the wound site, and
a control circuit coupled to said flow sensor and to said vacuum regulator for generating control signals for controlling said vacuum regulator, said control circuit generating an alarm signal if the flow rate sensed by said flow sensor exceeds a threshold; and
a wound dressing provided at the wound site and coupled to said vacuum regulator, said wound dressing comprising:
a wound dressing pad for placing over the wound, and
a wound drape provided over said wound dressing pad and the wound site for securing said wound dressing pad and sealing the wound site for application of the negative pressure.

16. The system of claim 15, wherein said control circuit comprises a processor, a display coupled to said processor, and at least one user interface switch coupled to said processor.

17. The system of claim 15, and further comprising a transmitter for transmitting information from said suction regulator to a healthcare facility records database.

18. The system of claim 17, wherein the information transmitted by said transmitter includes any one or more of the following: times at which negative pressure was applied to the wound, a pressure applied, intermittence cycles, times at which settings were changed along with new settings, leak detection alarm times, full canister alarms times, and readings from a flow sensor.

19. The system of claim 7, wherein said transmitter is a wireless transmitter for transmitting the information wirelessly to the healthcare facility records database.

20. The system of claim 15, wherein the vacuum regulator is coupled to a built-in vacuum system of a healthcare facility.

21. The system of claim 15, wherein said vacuum regulator is an internal vacuum pump.

22. A system for the treatment of wounds by applying a negative pressure to a wound site, the system comprising:
a suction regulator comprising:
a vacuum regulator for supplying a negative pressure to the wound site, and
a control circuit coupled to said vacuum regulator for generating control signals for controlling said vacuum regulator to selectively supply the negative pressure to the wound site; and
a wound dressing provided at the wound site and coupled to said vacuum regulator, said wound dressing comprising:
a wound dressing pad for placing over the wound, and
a wound drape provided over said wound dressing pad and the wound site for securing said wound dressing pad and sealing the wound site for application of the negative pressure,
wherein said wound dressing pad comprises a support material at least partially covered with silicone.

23. The system of claim 22, wherein said support material comprises at least one of a natural fiber, a polymer, and a foam.

24. The system of claim 22, wherein said wound dressing pad is coated with a solution that promotes healing of the wound.

25. The system of claim 22, wherein said wound dressing pad is coated with a solution that reduces adhesion of tissue and fluids to said wound dressing pad.

26. The system of claim 22, wherein said wound dressing pad is coated with a solution comprising at least one of polypropylene, glycol, saline, silver, and an anti-bacterial solution.

27. A wound dressing pad for use in a system for the treatment of wounds by applying a negative pressure to a wound site, said wound dressing pad comprising a support material at least partially covered with silicone for application to a wound at the wound site.

28. The wound dressing pad of claim 27, wherein said support material comprises at least one of a natural fiber, a polymer, and a foam.

29. The wound dressing pad of claim 27, wherein said support material is impregnated with the silicone.

30. The wound dressing pad of claim 27, wherein the silicone is applied to a surface of said support material that contacts the wound.

31. The wound dressing pad of claim 27, wherein the silicone is integral with said support material.

32. The wound dressing pad of claim 27, wherein the silicone forms a layer on a surface of said support material adjacent the wound.

33. The wound dressing pad of claim 32, wherein said silicone layer is slit to allow air to pass to and from the wound.

34. The wound dressing pad of claim 27, wherein said wound dressing pad is coated with a solution that promotes healing of the wound.

35. The wound dressing pad of claim 27, wherein said wound dressing pad is coated with a solution that reduces adhesion of tissue and fluids to said wound dressing pad.

36. The wound dressing pad of claim 27, wherein said wound dressing pad is coated with a solution comprising at least one of polypropylene, glycol, saline, silver, and an anti-bacterial solution.
